(19) United States
(12) Patent Application Publication
(10) Pub. No.: US 2019/0015058 A1
(43) Pub. Date: Jan. 17, 2019

(54) Mobile Molecular Imaging System and Intervention System Including Same

(71) Applicant: General Equipment for Medical Imaging, S.A., Valencia (ES)

(72) Inventors: Jorge ÀLAMO VALENZUELA, Valencia (ES); Carlos CORRECHER SALVADOR, Valencia (ES); Julio BARBERÁ BALLESTER, Valencia (ES); José MARTÍNEZ BENEDICTO, Valencia (ES)

(21) Appl. No.: 16/110,485

(22) Filed: Aug. 23, 2018

Related U.S. Application Data
(63) Continuation of application No. PCT/ES2017/070099, filed on Feb. 22, 2017.

(30) Foreign Application Priority Data
Feb. 26, 2016 (ES) P201630226

(51) Int. Cl. A61B 6/00; A61B 6/03; G06T 11/00; A61B 10/04; A61N 5/10; A61N 7/00; A61B 34/20

(52) U.S. Cl. CPC A61B 6/4405; A61B 6/037; A61B 6/4266; G06T 11/003; A61B 10/04; A61N 1/40; A61N 7/00; A61B 34/20; A61B 6/4476; G06T 2211/428; A61N 2005/1087; A61N 5/1001

(57) ABSTRACT

The present invention refers to a Mobile Molecular Imaging System, for example a PET (positron emission tomography) or SPECT (single photon emission computerized tomography) device, comprising:
at least one group of n detector modules conforming detector module sets with a rounded shape comprising m detector modules each, being m<n and said detector module sets being separable from each other,
first means to carry out the movement for separating the detector module sets in the transaxial plane
second means to carry out a rotation movement of the Imaging System
a Vertical Actuator that allows the axial movement of the Imaging System.
   and also refers to an Interventional System comprising the Mobile Molecular Imaging System and use of any of them in medical procedures, such as biopsy, radiotherapy, radiofrequency or ultrasound, among others.

### CROSS-REFERENCE TO RELATED APPLICATIONS

This application is a continuation of and claims priority to PCT Application No. PCT/ES2017/070099, filed on Feb. 22, 2017, which, in turn, claims priority to Spanish Application No. P201630226, filed on Feb. 26, 2016. The entire contents of each of these applications is incorporated herein by reference.

### FIELD OF THE INVENTION

The present invention is encompassed in the field of medical procedures using molecular imaging techniques and more particularly to interventional devices associated and guided by a molecular imaging system, such as a biopsy, radiofrequency, ultrasound or radiotherapy guided devices among others.

### BACKGROUND OF THE INVENTION

Imaging techniques applied in medicine envision the inside of living organisms allowing to perform accurate and early diagnosis that substantially affect the strategies in therapy, what improves the outcome of treatment and reduces the mortality and morbidity of multiple diseases. Its significance lies in several areas of health and research such as cancer diagnosis and staging, evaluation of the cardiovascular system and multiple applications in neuroscience and molecular genetics.

In this context, thanks to its inherent molecular characteristics (capable to observe nano-molar to pico-molar concentrations) and its excellent sensitivity, Nuclear Medicine plays an important role in providing functional information in three-dimensional in vivo (3D) on the biodistribution of a molecular tracer labeled with radioactive isotopes (i.e. a radiopharmaceutical) administered to the subject of study.

Currently, nuclear medicine is a clinical instrument widely used in oncology, cardiology and neurology. Mostly commercial equipment allows full-body imaging of patients, although equipment has recently appeared devoted to obtain images of specific organs.

A current limitation of nuclear medicine techniques is its unique diagnostic application because the equipment presents geometries hardly compatible with any type of intervention in the patient during the scan. Neither the full body equipment nor the dedicated organ equipment with closed configurations have the capacity to observe any intervention on the patient in real time.

There are medical imaging techniques that do allow viewing or tracking interventions, but are limited to structural imaging such as CT (computerized tomography), MR (magnetic resonance) or ultrasounds, wherein one can observe the morphology of the organ to be intervened, but not the biological functionality that a nuclear medicine system would be able to envision. Many of these systems also do not allow real-time monitoring, but only taking pictures at different times of the process to ensure that the intervention is carried out at the desired morphological point. Ultrasound based systems are an exception, allowing real-time images, but very low resolution and specificity.

For example during tumor biopsies guided by MR, common in clinical practice, the practitioner obtains an image of the patient within a full body equipment, by means of which the point to be biopsied is determined. The patient should be moved away from the equipment using a motorized stretcher to introduce a guide in the patient at the desired biopsy point and re-enter the patient in the equipment to obtain a confirmation image. After the confirmation the patient is moved again out of the equipment and the biopsy is carried out.

### BRIEF DESCRIPTION OF THE INVENTION

The subject matter disclosed herein describes a Mobile Molecular Imaging System, for example, a PET (positron emission tomography) or SPECT (single photon emission computerized tomography) device. The Mobile Molecular Imaging System includes: at least one group of n detector modules conforming detector module sets with a rounded shape comprising m detector modules each, being m<n and said detector module sets being separable from each other, a first means to carry out the movement for separating the detector module sets in the transaxial plane, a second means to carry out a rotation movement of the imaging System, and a Vertical Actuator that allows the axial movement of the Imaging System. The subject matter herein also refers to an Interventional System comprising the Mobile Molecular Imaging System and use of any of them in medical procedures, such as biopsy, radiotherapy, radiofrequency or ultrasound, among others.

These and other objects, advantages, and features of the invention will become apparent to those skilled in the art from the detailed description and the accompanying drawings. It should be understood, however, that the detailed description and accompanying drawings, while indicating preferred embodiments of the present invention, are given by way of illustration and not of limitation. Many changes and modifications may be made within the scope of the present invention without departing from the spirit thereof, and the invention includes all such modifications.

### BRIEF DESCRIPTION OF THE DRAWINGS

Various exemplary embodiments of the subject matter disclosed herein are illustrated in the accompanying drawings in which like reference numerals represent like parts throughout, and in which:

FIG. 1A represents a particular embodiment of a closed 3D configuration of the Imaging System of the invention, a PET device, with one group of detector modules composed of two sets of detector modules having semi-ring shape, according to the invention.

FIG. 1B represents a perspective view from top of the same closed 3D configuration of the PET device shown in FIG. 1A.

FIG. 2 represents a front view of the closed 3D configuration of the same embodiment as FIG. 1A and FIG. 1B.

FIG. 3 represents a top view of the closed configuration of the same embodiment as FIG. 1A and FIG. 1B.

FIG. 4 represents a front view of the 3D open configuration of the same embodiment as FIG. 1A and FIG. 1B.

FIG. 5 represents a front view of the complete 3D open turned with respect to the configuration shown in FIG. 4, corresponding to the same embodiment as FIG. 1A and FIG. 1B.

FIG. 6 represents a front view of the 3D open configuration (down) of the same embodiment as FIG. 1A and FIG. 1B.

FIG. 7 represents an example of an Imaging System, a PET, with two superposed detector groups, each of these groups being composed of two sets of semi-ring shaped detector modules, it specifically shows a front view of the complete 3D Image System corresponding to the same embodiment as FIG. 1A and FIG. 1B, but in an open configuration.

FIG. 8 represents a front view of the complete 3D open corresponding to the same embodiment as FIG. 1A and FIG. 1B.

FIG. 9 represents a front view of the complete 3D open (down) corresponding to the same embodiment as FIG. 1A and FIG. 1B.

FIG. 10 represents a front view of the open configuration corresponding to the same embodiment as FIG. 1A and FIG. 1B.

FIG. 11 represents a top view of the open configuration corresponding to the same embodiment as FIG. 1A and FIG. 1B.

FIG. 12: represents an Imaging Device, a PET, wherein a support to hold a patient's organ, body part, can be seen, for example a couple of pallets to hold a breast during a biopsy procedure, and the needle of a biopsy device associated to the PET.

FIG. 13. (A to E) Represents a chronological sequence of positions of an example of Interventional System of the invention during its use.

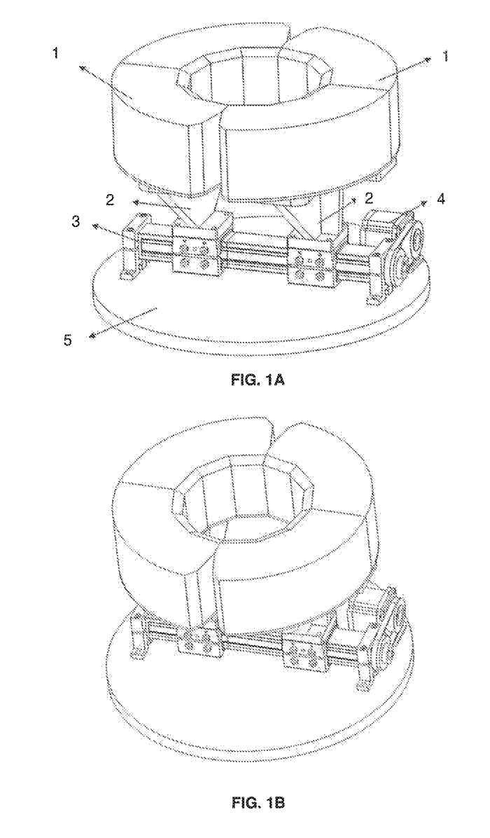

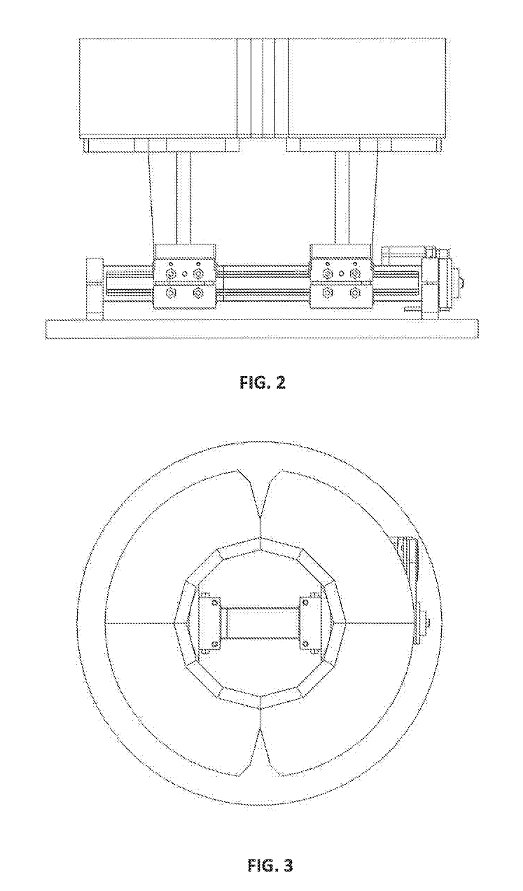

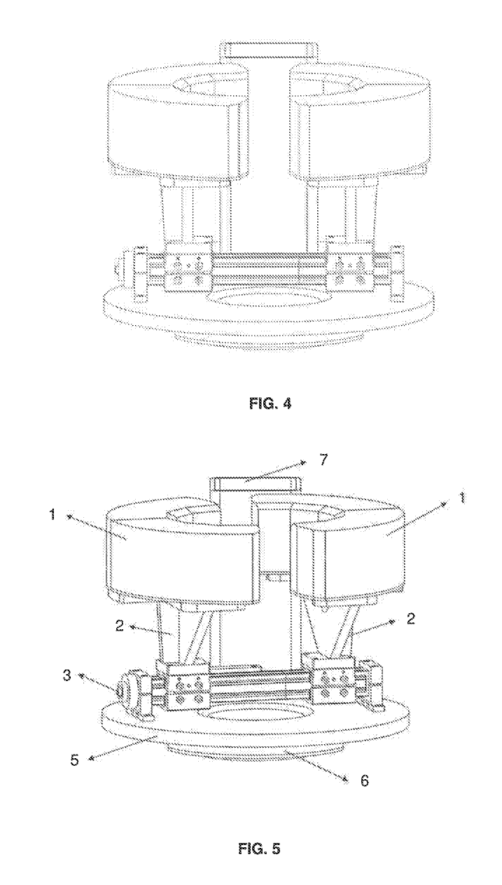

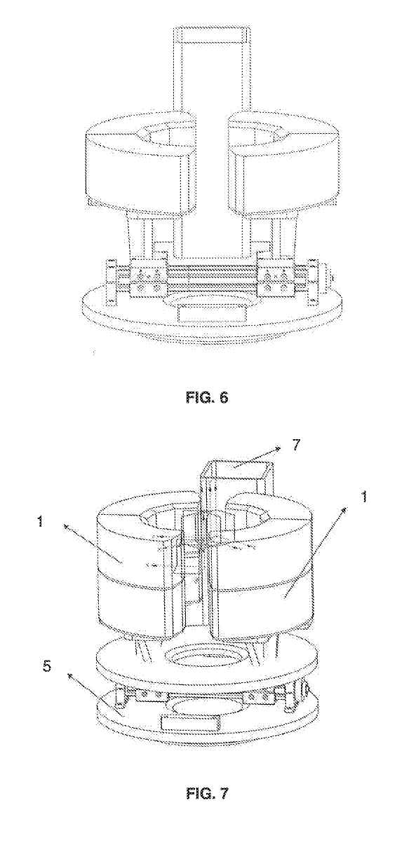

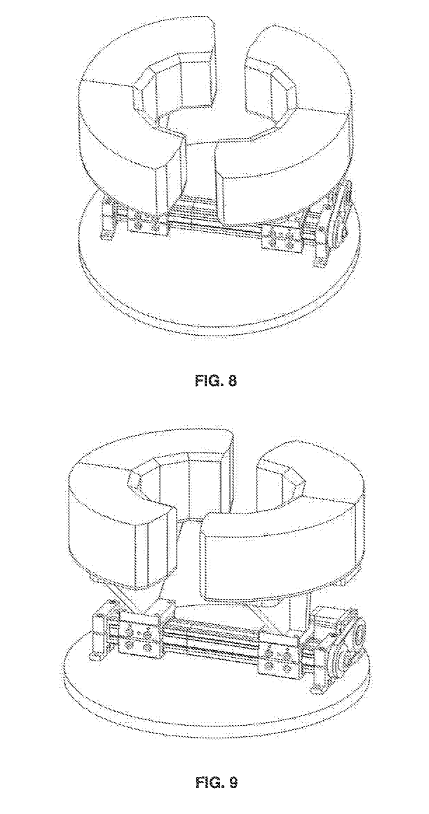

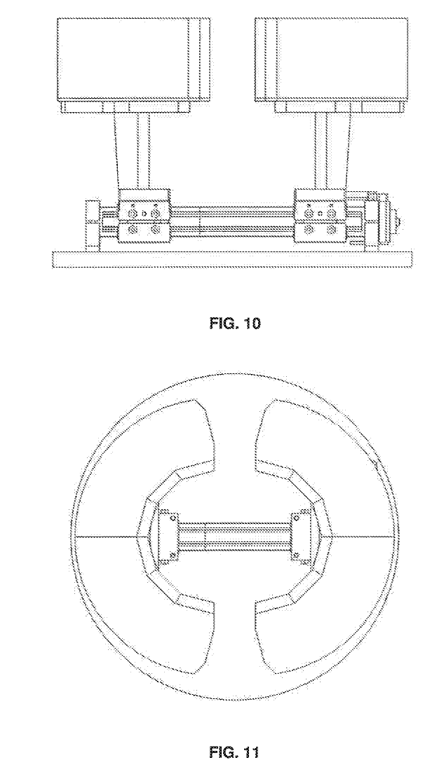

In describing the preferred embodiments of the invention which are illustrated in the drawings, specific terminology will be resorted to for the sake of clarity. However, it is not intended that the invention be limited to the specific terms so selected and it is understood that each specific term includes all technical equivalents which operate in a similar manner to accomplish a similar purpose. For example, the word “connected,” “attached,” or terms similar thereto are often used. They are not limited to direct connection but include connection through other elements where such connection is recognized as being equivalent by those skilled in the art.

### DETAILED DESCRIPTION OF THE INVENTION

### Definitions and Abbreviations Used in this Specification

“Mobile Molecular imaging System”: it refers to a system for obtaining images using radiation, for example gamma radiation, it is abbreviated as “Imaging System”.

“interventional Device”: it refers to a device or apparatus intended to carry out an intervention in a patient's organ or in the patient's body, such as a biopsy, radiofrequency, radiotherapy or ultrasound device, among others, and that is associated to the Mobile Molecular imaging System.

“Semi-ring”: here it is a set of detector modules with a semi-circumference or semi-ellipse shape or also called “C”-shape. In case of a PET (positron emission tomography) device it can also be called a PET semi-ring” or “PET group”.

“Interventional System”: it refers to a System that combines the Mobile Molecular Imaging System” and the Interventional Device.

“Associated” means that the Interventional System can be mechanically linked to the Mobile Molecular Imaging System or can just be guided by the Mobile Molecular Imaging System through the use of a common reference coordinates system.

“Biopsy System”, “Biopsy Guided Set” and “Biopsy Device” are expressions used indistinctly.

“Interventional process” and “medical procedure” are expressions used indistinctly and refer to a series of steps directed to interfere with the outcome or course especially of a condition or process—as to prevent harm or improve functioning—,

“Intervening tool”: it refers to any instrument or tool to be used in the medical procedure. “Axial”, unless otherwise indicated, refers to the “Z” axis.

The present invention relates to a Mobile Molecular Imaging System comprising:
at least one group of n detector modules conforming detector module sets with a rounded shape comprising m detector modules each, being m<n and said detector module sets being separable from each other,
first means to carry out the movement for separating the detector module sets in the transaxial plane
second means to carry out a rotation movement of the Imaging System
a Vertical Actuator that allows the axial movement of the Imaging System.

According to particular embodiments the rotation movement of the Imaging System is a rotation around an axial axis—usually Z axis—of the detector module sets.

In the Imaging System of the invention, according to additional particular embodiments:
the first means are:
   first mechanical supports to carry out the movement for separating the detector module sets in the transaxial plane
      an horizontal actuator to move the mechanical supports in opposite directions and
      a manual handle associated to the horizontal actuator
and said second means are:
   a rotating mechanical support to carry out the rotation movement of the Imaging System around an axial axis
   a manual handle associated to the rotating mechanical support.

In the Imaging System of the invention, according to additional particular embodiments:
the first means are:
   first mechanical supports to carry out the movement for separating the detector module sets in the transaxial plane
      an horizontal actuator to move the mechanical supports in opposite directions and
   a motor associated to the horizontal actuator
and said second means are:
   a rotating mechanical support to carry out the rotation movement of the Imaging System
   a motor associated to the rotating mechanical support.

According to more particular embodiments the second means are
a rotating mechanical support to carry out the rotation movement of the Imaging System around an axial axis—usually Z axis—of the detector module sets and
a motor associated to the rotating mechanical support.

According to particular embodiments the sets of m detector modules have the form of a ring fragment, including the possibility of being an ellipse fragment, and preferably, they are semi-ring shaped, semi-ellipse shaped or C-shaped structures, comprising n/2 detector modules each.

An Imaging System can comprise one or more groups of detector modules, for example it can comprise two superposed groups, each of these groups being composed of at least two sets of detector modules.

The Mobile Molecular Imaging System can be selected between a SPECT (single photon emission computerized tomography) and a PET device, and more preferably it is a PET device.

The Imaging System according to particular embodiments is a PET device or a SPECT device, preferably a PET device, wherein the detector modules can have the features of conventional devices with regard to the type of crystals. For example, they can comprise monolithic or pixelated scintillation crystals, as any conventional device. Any type of continuous scintillation crystal known in the art can be used as regard to shape and composition. The monolithic scintillation crystals, for example LYSO (lutetium-yttrium oxyorthosilicate), can be in particular embodiments, trapezoidal monolithic scintillation crystals. The scintillation crystals can be coupled to any known and appropriate photomultiplier or photomultiplier array, for example PSPMTs (position sensitive photomultipliers tubes).

The Imaging System can rotate up to 360 degrees and it can be placed in a configuration wherein the sets, preferably semi-rings of detector modules, are separated up to a distance equivalent to the semi-ring diameter (diameter of the ring formed by the two semi-rings), or to the major diameter in the case of detector sets detector groups with an ellipse form.

According to particular embodiments the Imaging System rotates up to 180° and it can be placed in a configuration wherein the sets, preferably semi-rings of detector modules, are separated up to 100 mm.

The present invention also refers to an Interventional System comprising the Imaging System described above and a Interventional Device, which is as defined above, a device or apparatus intended to carry out an intervention in a patient's organ or patient's body and that is associated to the Mobile Molecular Imaging System. The term “associated” has the meaning given above: this is that the Imaging System can be mechanically linked to the Mobile Molecular Imaging System or can just be guided by the Mobile Molecular Imaging System through the use of a common reference system, that can be placed, for example, in a biopsy grid, in the case of a biopsy device.

Particular embodiments of the Interventional Device refer to a radiotherapy device, a radiofrequency device, a brachytherapy device, a protontherapy device, an ultrasound device, a HIFU (high intensity focused ultrasound) or a biopsy (or surgical) guided set.

The Interventional System, according to preferred embodiments, includes a first processor device capable to reconstruct the information acquired by the Imaging System into three dimensional images of the distribution of a radiotracer in the field-of-view. This processor is capable to reconstruct images at any location of the Imaging System in every configuration (open, closed, rotated, etc. . . . ).

The first processor device is capable to reconstruct images with a refreshing rate sufficient to guide the interventional process (for example, for a particular embodiment referring to a biopsy device, 2 images per second could be adequate).

The Interventional System, according to preferred embodiments, includes a second processor device capable to calculate the optimal trajectory of the Interventional Device in the patient body or organ, based on one location, for example the lesion to biopsy (selected by the user) in the image obtained by the first processor. The trajectory is restricted to the available positions achievable by the Interventional System.

The Interventional System can further include a support to hold the desired organ or body part of the patient during the intervention to avoid undesired movements of the organ. For example two pallets can hold the organ, for example a breast, with soft compression during the intervention or procedure.

The Interventional System can be handled manually or can be handled through actuators, mechanically linking the Imaging System to the interventional device.

In the particular case of a Biopsy guided set, it can comprise several mechanical actuators, which allow a needle to move in the three spatial directions. The Imaging System is compatible with a commercial biopsy system. The Imaging System can also include a laser device which can locate the tip of the needle to ensure the precise localization of the needle before starting the procedure.

Particular embodiments of a Biopsy Guided Set are a biopsy device associated to a PET device, and a biopsy device associated to a SPECT device.

The invention also refers to the use of the Imaging System or the use of the Interventional System, for example a Biopsy Guided Set described above, for carrying out an intervention in a patient's body, for example a biopsy procedure.

The use of the Interventional System of the invention comprises the following steps:
a first step consisting of obtaining first images with the Imaging System,
a second step, comprising moving the Imaging System in the axial direction positioning a first detector module set with the appropriate trajectory path for an intervening tool,
a third step, wherein the detector module sets, such as detector module sets with a semi-ring shape, are fixed and the interventional device is operated.

The Interventional System can comprise processor device that generates images during a medical procedure or interventional process. Said images can further be generated during the interventional process in real time.

Particular embodiments of the use of the Interventional System of the invention comprises the following steps:
a first step consisting of making first images such as an initial scan of a body part/organ to be intervened.
a second step, comprising moving the Imaging System in the axial direction, positioning the upper groups of semi-rings in a a PET with two superposed detector module groups, in the region of interest selected, rotating the first detector module semi-rings, to align a C ring aperture, with the appropriate trajectory path for the intervening tool, for example a needle, and making new scans,
a third step, wherein the semi-rings are fixed to avoid any rotation or movement during the intervention, for example a biopsy procedure, and carrying out the intervention, for example a biopsy.

The images are obtained at several steps to ensure the correct location of the intervening tool, for example a needle, during the intervention.

The third step further comprise performing short acquisitions of images (around 2 minutes) and correcting the orientation of the intervening tool, for example a needle, if it is necessary.

The process of using the Interventional System of the invention can refer in a particular case, to the use of an Interventional Device with its own mechanical properties that make it capable of placing itself in the desired position, for example a Biopsy Guided Set combined with a PET device. The PET device can comprise two superposed sets of semi-rings of detector modules (as in FIG. 7) that can be separated mechanically in order to allow the needle insertion. The first acquisition of images of the patient organ, for example breast, is performed with the closed ring configuration in order to obtain a high quality image to locate the lesion. Then, the software calculates the optimum path for the biopsy and moves the biopsy and PET systems to the desired position. At this point, two compression pallets are used to hold the breast. Then, the PET system opens and the biopsy procedure starts.

According to particular embodiments of a biopsy guided device a vertical elevator, or vertical actuator, can be provided that jointly moves the detectors of the Imaging System and the biopsy device in order to allow the biopsy needle to locate in the optimum axial position to start the procedure.

The Imaging System of the invention is able to perform high-quality images in its closed configuration and present the ability to separate the detectors to allow the biopsy needle to enter in the FOV. Although the image quality with the open configuration degrades comparing with the closed configuration, it is more than sufficient to perform the biopsy procedure as the image is just used as a guidance to locate the lesion,

### REFERENCES USED IN THE FIGURES

1. PET Semi-ring/PET Group
2. Mechanical support to carry out the movement for separating the two semi-rings
3. Horizontal actuator to carry out the movement for separating the two semi-rings. It allows that each of the two mechanical supports goes in an opposite direction
4. Motor of the horizontal actuator
5. Mechanical support to carry out the rotation movement of the assembly
6. Motor of the rotation mechanical support
7. Vertical Actuator that allows the movement of the assembly with regard to that axis.
8. Needle in a biopsy device associated to a PET device
9. Support for holding the patient's organ or body part,

### Example

A particular embodiment of the Molecular Imaging System is a PET device as the one shown in FIG. 7, comprising two superposed sets of semi-rings of detector modules (two upper semi-rings and two lower semi-rings) with the same number of detector modules each semi-ring, preferably 6 detector modules each.

According to this specific embodiment a PET system is provided consisting of two rings with 12 modules each. Each module contains a single LYSO continuous scintillation crystal coupled to a PSPMT H8500 from Hamamatsu Photonics (Hamamatsu city, Japan) and an electronic readout board. The use of trapezoidal crystals reduces the image compression effect and improves energy, spatial, and depth of interaction (DOI) resolutions especially when considering truncation angles smaller than 60°. The Mobile Molecular Imaging System—here a PET with detector modules forming semi-rings, has an aperture of 186 mm.

The detector design uses 12 mm thick scintillation LYSO crystals whose front and back face are 40×40 mm² and 50×50 mm² respectively. The back face of crystals is polished and is 0.25 mm separated from the PSPMTs. All the other crystal surfaces, are roughened and painted black.

The image acquisition system (semi-rings+electronics+computer) is composed of a trigger card, responsible for detecting the coincidence events, and several separated A/D conversion cards. The conversion is started by the trigger card when two events are detected in a time window of 5 ns. A back plane electronic board connects trigger and A/D cards and routes the signaling.

Coincidences between each detector and its seven opposite detector modules are allowed, providing a transaxial FOV (field-of-view) of 170 mm. The axial FOV covers 94 mm. A precise vertical elevator jointly moves the detector and the biopsy device in order to allow the biopsy needle to locate in the optimum axial position to start the procedure.

The biopsy device is composed by several mechanical actuators, which allow a needle to move in the three spatial directions. The system is compatible with commercial biopsy systems It also includes a laser device which can locate the tip of the needle to ensure the precise localization of the needle before starting the biopsy procedure.

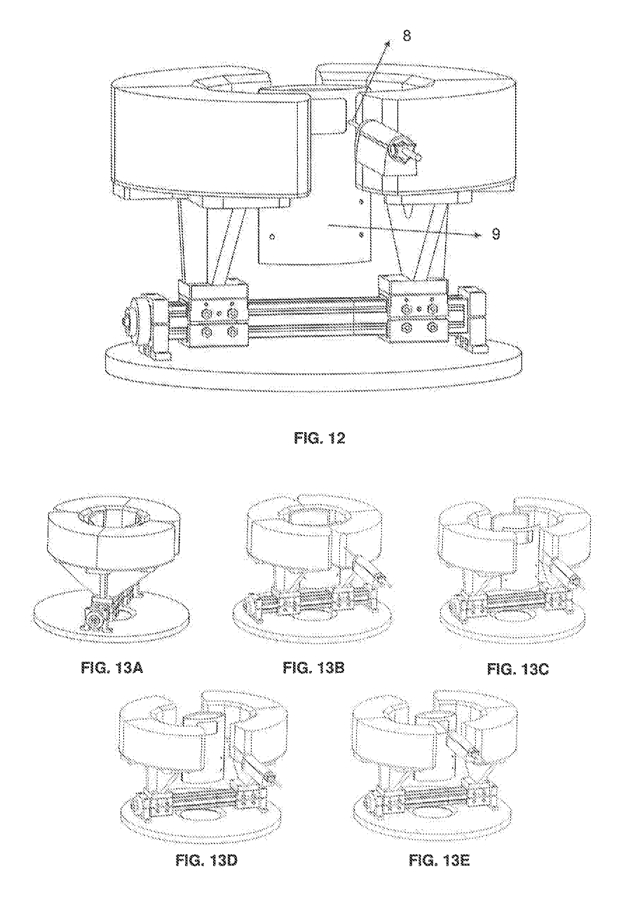

To enable the biopsy procedure, the detectors of the PET are separable up to 60 mm in the transaxial direction becoming two groups of 6 detectors forming a “C” shape, allowing the passage of the needle (FIG. 12). In addition, the system can rotate up to 170 degrees to position the detector semi-rings in the optimum location to minimize the biopsy trajectory to the lesion. The system includes two pallets to hang hold the breast during the biopsy procedure.

The defined protocol to perform a breast exploration with biopsy consists in three steps each one corresponding to a different position of the PET device (FIG. 13). The first step consists of an initial scan of the full breast with the closed detector configuration, generating a high quality image in order to determine the lesion location. In the second step, the detector system moves in the axial direction positioning the upper ring in the region of interest selected. Then, the detector rings rotate to align the C ring aperture with the shortest trajectory path in order to introduce the needle in the breast. The rings open and the pallets fix the breast. The detector system performs new acquisition and relocates on the 3D image the location of the lesion, as the pallet compression should move the original lesion location. On last step, the ring is fixed to avoid any rotation or movement during the medical procedure. The biopsy starts and the system performs short acquisitions (around 2 minutes) for tracking the lesion position and the needle, making several steps to reach the final location, always supervised and controlled by the user. The needle orientation can be corrected in each step if it is necessary.

An examination table is provided, where the patient is situated in prone position. A PET system according to the invention is used and complete biopsy device. Two transparent polycarbonate pallets (FIG. 12) are provided to hold the breast during the biopsy procedure and all the engines required to move the different components, including the aperture and rotation of the PET semi-rings.

In the posterior area of the device the PET acquisition computer and the electrical and electronic components are located. The acquisition and reconstruction software are located in a separated portable cart, which includes a—workstation with a data storage system, two monitors and a medical grade keyboard.

1. A Mobile Molecular Imaging System comprising:
at least one group of n detector modules conforming detector module sets with a rounded shape comprising m detector modules each, being m<n and said detector module sets being separable from each other;
a first means to carry out the movement for separating the detector module sets in the transaxial plane;
a second means to carry out a rotation movement of the Imaging System around an axial axis, such that said second means include:
a rotating mechanical support to carry out the rotation movement of the Imaging System around the Z axis of the detector module sets, wherein the rotating mechanical support rotates up to 180 degrees, and
a motor associated to the rotating mechanical support; and
a Vertical Actuator that allows the axial movement of the Imaging System, wherein said system is a PET device and wherein the sets of n detector modules are ring-shaped and are forming structures that are semi-rings comprising n/2 detector modules each.

2. The Imaging System of claim 1 wherein,
the first means comprise:
first mechanical supports to carry out the movement for separating the detector module sets in the transaxial plane,
a horizontal actuator to move the mechanical supports in opposite directions, and
a manual handle associated to the horizontal actuator; and
the second means comprise:
a rotating mechanical support to carry out the rotation movement of the Imaging System around an axial axis, which is the Z axis of the detector module sets, and
a manual handle associated to the rotating mechanical support.

3. The Imaging System of claim 1 wherein,
the first means comprise:
first mechanical supports to carry out the movement for separating the detector module sets in the transaxial plane,
a horizontal actuator to move the mechanical supports in opposite directions, and
a motor associated to the horizontal actuator; and
the second means comprise:
a rotating mechanical support to carry out the rotation movement of the Imaging System around an axial axis, and
a motor associated to the rotating mechanical support.

4.-8. (canceled)

9. The Imaging System of claim 1, further comprising a first processor device capable to reconstruct information acquired by the Imaging System into three dimensional images of the distribution of a radiotracer in a field-of-view.

10. An Interventional System comprising:
a Mobile Molecular Imaging System comprising:
at least one group of n detector modules conforming detector module sets with a rounded shape comprising m detector modules each, being m<n and said detector module sets being separable from each other,
a first means to carry out the movement for separating the detector module sets in the transaxial plane,
a second means to carry out a rotation movement of the Mobile Molecular Imaging System around an axial axis, such that said second means include:
a rotating mechanical support to carry out the rotation movement of the Mobile Molecular Imaging System around the Z axis of the detector module sets, wherein the rotating mechanical support rotates up to 180 degrees, and
a motor associated to the rotating mechanical support,
a Vertical Actuator that allows the axial movement of the Mobile Molecular Imaging System wherein said system is a PET device and wherein the sets of n detector modules are ring-shaped and are forming structures that are semi-rings comprising n/2 detector modules each.

11. The Interventional System of claim 10, further comprising:
a first processor device capable to reconstruct information acquired by the Imaging System into three dimensional images of the distribution of a radiotracer in a field-of-view; and
a second processor device capable to calculate an optimum trajectory for an interventional device, based on the image generated by the first processor and a selected region-of-interest.

12. The Interventional System of claim 10, further comprising a support to hold an organ or body part of the patient during an intervention.

13. The Interventional System of claim 10, wherein the Mobile Molecular Imaging System is linked to the interventional device via one of a manual actuator and a motor driven actuator.

14. The Interventional System of claim 10, selected from one of a radio therapy device, a proton therapy device, a brachytherapy device, a radiofrequency device, a Biopsy Guided device, a ultrasound device and a high frequency ultrasound device (HIFU).

15. A method for carrying out a medical procedure using a Mobile Molecular Imaging System, wherein the Mobile Molecular Imaging System comprises:
at least one group of n detector modules conforming detector module sets with a rounded shape comprising m detector modules each, being m<n and said detector module sets being separable from each other,
a first means to carry out the movement for separating the detector module sets in the transaxial plane,
a second means to carry out a rotation movement of the Mobile Molecular Imaging System around an axial axis, such that said second means include:
a rotating mechanical support to carry out the rotation movement of the Mobile Molecular Imaging System around the Z axis of the detector module sets, wherein the rotating mechanical support rotates up to 180 degrees, and
a motor associated to the rotating mechanical support,
a Vertical Actuator that allows the axial movement of the Mobile Molecular Imaging System, wherein said system is a PET device and wherein the sets of n detector modules are ring-shaped and are forming structures that are semi-rings comprising n/2 detector modules each, the method comprising the steps of:
obtaining a plurality of first images with the Mobile Molecular Imaging System:
moving the Mobile Molecular Imaging System in at least one of an axial direction and a rotational direction to position a first detector module set with a trajectory path for an intervening tool; and
fixing the position of the first detector module set during operation of the intervening tool.

16. The method of claim 15, wherein a first processor of the Mobile Molecular Imaging System generates images during operation of the intervening tool.

17. The method of claim 16, wherein a first processor of the Mobile Molecular Imaging System generates images during operation of the intervening tool in real time.
